(19) United States
(12) Patent Application Publication
(10) Pub. No.: US 2010/0100004 A1
(43) Pub. Date: Apr. 22, 2010

(54) Skin Temperature Measurement in Monitoring and Control of Sleep and Alertness

(75) Inventor: Eugenio Johannes Wilhelmus van Someren, Amsterdam (NL)

Correspondence Address: THE WEBB LAW FIRM, P.C., 700 KOPPERS BUILDING, 436 SEVENTH AVENUE, PITTSBURGH, PA, 15219, US

(73) Assignee: Koninklijke Nederlandse Akademie van Wetenschappen, Amsterdam (NL)

(21) Appl. No.: 12/335,269

(22) Filed: Dec. 15, 2008

(30) Foreign Application Priority Data
Oct. 16, 2008 (EP) 08166782.6

(51) Int. Cl. A61B 5/01; A61B 5/103

(52) U.S. Cl. 600/549; 600/595

(57) ABSTRACT

Method of an arrangement for monitoring sleep in a subject by measuring within a prescribed interval skin temperature of a predetermined region of the subject's body and a motion sensor for sensing motion of the subject, comparing the measured skin temperature of the predetermined region with a predetermined temperature threshold, and classifying the subject as being asleep or awake based on whether the skin temperature of the predetermined region is above or below the temperature threshold and on the motion data. In alternative aspects the invention relates to methods of and arrangements for manipulating sleep, as well as monitoring or manipulating alertness.

### FIELD OF THE INVENTION

The present invention concerns methods of monitoring sleep and/or level of alertness of a subject and to a system that can be used in such methods. Moreover, the invention relates to methods and arrangements for control of sleep and/or alertness.

### BACKGROUND OF THE INVENTION

The present invention relates to methods and arrangements for measuring and, optionally, controlling a condition of a human body, the condition being either one of being asleep, being awake but non-alert, and being alert. The condition is monitored by measuring skin temperature on one or more predetermined parts of a subject's body depending on the exact condition to be measured. Moreover, the condition may be manipulated by controlling skin temperature on one or more, possibly other, predetermined parts of the subject's body.

### Sleep.

Optimal human functioning requires sound sleep during the night. With advancing age, an increasing number of people complain about their sleep quality (Foley et al., 1995; Kryger et al., 2004). Nocturnal awakenings occur more frequently, especially in the morning, and the time spent in slow wave sleep decreases.

Sleep problems affect more than 70 million people in the United States alone. The majority of sleep disorders go undiagnosed and untreated. Sleep disorders range from snoring to insomnia, and can be as serious as obstructive sleep apnea, when a person stops breathing multiple times during the night.

Without proper sleep, people: (a) are more susceptible to illnesses and have a greater chance of experiencing emotional and mental health problems, (b) have a lower frustration tolerance and may tend to overreact when stresses occur in their lives, and (c) have diminished capacities to concentrate, remember, learn and complete tasks. Thousands die each year in automobile accidents that are the result of someone falling asleep at the wheel. There is even evidence that without proper sleep we accelerate aspects of the aging process and shorten our life span.

Although most are aware of the importance of a good night's sleep, there presently is no easy method of gauging just how well we actually do sleep. For most people, awareness of the quality of their sleep (i.e., how soundly they slept through the night) without a means of measurement is difficult, if not impossible. The importance of estimating total sleep time (TST) is underscored by recent studies showing clear associations of self-reported sleep duration with a variety of health outcomes, including diabetes, hypertension, coronary artery disease, and mortality.

Sleeping disorders are usually diagnosed only when a person goes to a sleep centre to have diagnostics taken over night. The golden standard for such diagnoses is polysomnography (PSG). Sensors have to be attached to several sites on the head and possibly other parts of the body. A patient spends around 45 minutes getting hooked up to a machine via electrodes, sensory belts, a microphone, and various other sensors. After the process, which is painless and made to be as non-intrusive as possible, the patient goes to sleep and the numbers are gathered. Recorded physiological signals need evaluation by trained sleep technicians supervised by a clinical neurophysiologist. All these requirements make it costly, difficult for patients under examination and consequently possible for only a limited number of days, usually one or two at most. There is no doubt that this method of sleep monitoring works, or that it is the main mode of diagnosing many important sleeping disorders. However, PSG has several drawbacks, including cost, time, and environment.

The majority of previous researches in the area of polysomnography technology has been directed toward the development hardware designs that would be more practical in a sleep center than at home. Measuring electroencephalography (EEG), electromyography (EMG) and electrooculography (EOG)—key parameters needed to assess sleep quality—is not easy. Taped-on electrodes are not only highly obtrusive and likely to keep the user awake, they are also prone to lose signal quality during prolonged registration or becoming dislodged or disconnected as users roll over in their sleep.

In order for a truly universal system for monitoring sleep and diagnosing disorders to become a reality, it must be moved from the professional sleeping center to the home. Although this is not a new idea, the technology has only recently caught up with such a proposal.

Actigraphy, which has often been applied as a proxy to PSG, is the continuous recording of body movements with a small integrated recorder, usually worn like a watch on the wrist. Subsequent analysis estimates epochs of sleep and wakefulness (usually at 30 sec or 1 min intervals) with an algorithm based on the likelihood that a person is asleep increases with prolonged inactivity. The actigraph is a small, wrist-worn device that contains an accelerometer to monitor the number of wrist movements per epoch (e.g., 30 or 60 sec). Scoring algorithms are used to identify sleep or wake states from activity counts and to determine sleep parameters such as sleep onset latency (SOL), total sleep time (TST), number and duration of awakenings, and sleep efficiency (SE=ratio of TST to total time in bed *100). Several types of devices that monitor activity have been used for clinical and research purposes (e.g., Actillume, Actiwatch, Gaëhwiler, MotionLogger). Each of these devices records activity in different ways and has unique algorithms for estimating sleep/wake variables from activity counts. The raw activity counts and sleep/wake measures from these systems may or may not be comparable.

Various algorithms have been proposed to score sleep/wake states using activity counts recorded by actigraph. Some use activity count thresholds to determine sleep/wake states. Others use statistical analyses to determine the actigraphy variables that predict sleep/wake in PSG, then use these variables to build regression equations to predict sleep/wake states. A comparison of certain algorithms developed by Cole R J, Kripke D F, Gruen W, Mullaney D J, Gillin J C. “Automatic sleep/wake identification from wrist activity”. Sleep. 1992; 15:461-69, and Sadeh A, Sharkey M, Carskadon M A. “Activity-based sleep-wake identification: an empirical test of methodological issues”. Sleep. 1994; 17:201-7. showed that both exhibit good sensitivity to detect sleep (>97%) but very low specificity (ability to detect wake), especially Cole's (34%). Using a commercial algorithm with different activity counts thresholds, Kushida C A, Chang A, Gadkary C, Guilleminault C, Carrillo O, Dement W C, “Comparison of actigraphic, polysomnographic, and subjective assessment of sleep parameters in sleep-disordered patients”. Sleep Med. 2001; 2:389-96, and Signal T. L., Gale J, Gander P. H., “Sleep measurement in flight crew: comparing actigraphic and subjective estimates to polysomnography”. Aviat Space Environ Med. 2005; 76:1058-63, showed that low thresholds are more specific (better able to detect wakefulness) while high-threshold algorithms are more sensitive (better able to detect sleep).

A number of studies have evaluated the ability of actigraphy to discriminate between sleep and wake as defined by PSG criteria. The majority of these were done using the Actillume device. In an extensive literature review of the role of actigraphy in sleep research it has been reported that actigraphy and PSG show overall minute-by-minute concordance rates of 91%-93% in adult populations. In a more recent review, Acebo C, LeBourgeois M K, “Actigraphy”. Respir Care Clin. 2006; 12:23-30 reported high epoch-by-epoch agreement rates (>85%) between actigraphy and PSG in healthy subjects of different age groups. However, these high overall concordance rates often mask the very low capacity of actigraphy to detect wake. Several studies using different types of devices have estimated actigraphy wake detection at around 35%-50%, a level equivalent to chance. In general, actigraphy tends to overestimate TST and SE, while underestimating sleep latency compared with PSG. These biases are not surprising, since actigraphy has difficulty detecting wake when the subject lies immobile in bed in a non-sleeping state. Actigraphy is much more affordable than PSG and easy for patients under examination. Multiple recording days are feasible. However, the validity of actigraphy is far from optimal. False positive wake classification is only a small problem (only sometimes movements occur during sleep). False positive sleep classification can be a severe problem though, all the more so with aging and insomnia; people can be awake for a considerable time without moving. Actigraphy in general appears to be less accurate in populations showing fragmented sleep compared to healthy subjects. This points to a very important weakness for the use of actigraphy as such in clinical populations or in situations where the sleep-wake cycle is challenged, such as jet lag and shift work.

Kräuchi et al., Nature (1999), 401, 36-37, discloses that vasodilation of distal skin regions was the best predictor of subsequent sleep onset latency. In this work, indications of vasodilation are provided by the distal-proximal temperature gradient an indirect index of distal heat loss. The research by Kräuchi et al. only concerns the predictive value of the distal-proximal temperature gradient to subsequent steep onset latency, not the determination of sleep/wake states throughout the entire period of ‘sleeping’. In addition Kräuchi et al. only describes the usefulness of the distal-proximal temperature gradient as an indicator of subsequent sleep onset latency, and not that of distal or proximal skin temperature alone. As noted before, the present inventors found that determinations of gradients do not outperform far more convenient single-site temperature measurements in discriminating false from true negative and false from true positive actigraphy classifications.

U.S. Pat. No. 5,441,476 discloses a body temperature regulation system for aiding a subject in falling asleep and assuring a good quality of sleep. The system is controlled on the basis of the subject's skin temperature. According to U.S. Pat. No. 5,441,476 the temperature of the skin surface becomes uniform, so as to reach the so-called basal temperature, as sleep progresses towards St. 3 (deep sleep), and the difference between the core temperature and the skin temperature is minimized. U.S. Pat. No. 5,441,476 teaches to measure the skin temperature of the thigh and torso while not providing any indication or suggestion to the effect that distal skin temperature measurements would constitute far better means to discriminate false from true actigraphy sleep classifications than do proximal skin temperature measurements. Moreover, the measurements as shown in U.S. Pat. No. 5,441,476 seem to show when sleep progresses the temperature of distal body parts like hands and feet lowers instead of increases.

Raymann et al., Skin deep: enhanced sleep depth by cutaneous temperature manipulation, Brain (2008), 131, 500-513, which is incorporated herein by reference, describe the effects of subtle skin temperature manipulations on sleep. According to Raymann et al. distal skin warming enhances REM sleep and suppresses light sleep in young and older subjects without sleep complaints whereas proximal warming results in deeper sleep and suppressed wakefulness. In elderly insomniacs proximal warming enhances slow wave and REM sleep whereas distal warming enhances slow wave sleep and suppresses REM sleep. Raymann et at only describe effects of skin temperature manipulations on sleep and not the effect of steep on skin temperature in a person not subjected to temperature manipulations. Moreover, Raymann et al. suggest to use a control loop in a bed in which temperature sensors are used to measure skin temperature and a heating source to manipulate the skins temperature in order to promote sleep. However, this temperature sensors are only used to measure the temperature and not to provide an indication whether or not the person is actually sleeping. Moreover, Raymann et al. do not suggest that to that effect one can best measure skin temperature at a distal part of the human body.

There is, therefore, a considerable need for a simple and reliable method and apparatus for sleep monitoring and/or controlling. Such methods and apparatuses should have the typical advantages of the known actigraphy systems of being suitable for home use and causing relatively little interference with a subjects normal sleeping habits, while having improved reliability, especially causing less false positive sleep determinations.

### Alertness.

At the other side of the spectrum of the condition of the human body opposite to being asleep is being alert. As used herein, the term “alert” refers to a state of a subject in which he/she is able to pay close and continuous attention. Monitoring of alertness may be of particular interest in subjects carrying out certain tasks that require a high amount of mental effort and organization, while such tasks involve certain risks, such as driving a vehicle or operating (heavy) machinery, but also performing purely mental or intellectual tasks, like monitoring dangerous chemical and nuclear plants. Alertness is related to psychology as well as physiology. People who (structurally) lack alertness may have narcolepsy, attention deficit disorder, chronic fatigue syndrome, depression, Addison's disease, or sleep deprivation.

Previously proposed methods for the continuous monitoring of alertness levels involved quantification of eyelid closure speed and frequency, head movements and electroencephalography. Methods of monitoring the ability for alert performance on the basis of skin temperature measurements, to the best knowledge of the inventors, have not been described in the art before. Of note, the predictive value of skin temperature measurements for alertness estimation cannot be derived from any previously published report on the predictive value of skin temperature for sleep onset latency, because human performance cannot be reliably inferred from measures of sleepiness like sleep onset latency tests (e.g. Lamond N, Jay S M, Dorrian J, Ferguson S A, Jones C, Dawson D. The dynamics of neurobehavioural recovery following sleep loss. J. Sleep Res. 2007;16(1): 33-41.

It is known that there is a relation between skin temperature and vigilance. Raymann et al., Sleep (2007) 30, 96-103, describe the effects of skin warming on vigilance. They observe that skin warming accelerates the decline in vigilance associated with the prolonged performance of a monotonous task. This document only deals with heating up the human body with an external heating source and does not deal with measurements on a human body that is not externally heated.

JP11042282 discloses a method to subtly cool the face without inducing so much cold that it activates thermoregulatory responses and thereby disturbs sleep or vigilance. This document deals with the state of a subject in the area between being awake (vigilant) or being in a hypnagogic state. This document does not deal with alertness as referred to here, i.e., a state in which a person is able to pay close and continuous attention.

There is a need for simple and easy to use methods of and equipment for monitoring and/or controlling alertness.

### SUMMARY OF THE INVENTION

The invention provides methods and arrangements for monitoring sleep and/or alertness as well as arrangements for stimulating sleep or alertness. Essentially, therefore, the invention relates to four different sub-aspects that can be summarized as follows:

1. the first aspect relates to monitoring sleep,

2. the second aspect relates to monitoring alertness,

3. the third aspect relates to stimulating sleep, and

4. the fourth aspect relates to stimulating alertness.

As will become apparent from the description below, in some embodiments these aspects are combined. Yet, they will be dealt with separately.

First aspect: monitoring sleep.

A method according to the invention that meets the needs of the first aspect is defined in independent claim 1. Moreover, a device that is arranged to perform such a method is defined in independent claim 7.

The present inventors, as a result of thorough research, have discovered that an easy to use temperature indicator of whether a person is asleep or awake is the temperature of a distal part of the human body.

The inventors have found that an extremely good indicator of being asleep or awake is based on combining actigraphy measurements with measurements of the skin temperature, preferably, of distal body parts. Motor activity of the subject may be measured by sensing temporal motion of at least one part of the subject's body, comprising at least one of a part of a subject's arm, a part of a leg, and a wrist. More in particular, and as will be illustrated in the examples hereinafter, the inventors have shown that distal skin temperature can be used to improve the discrimination of false from true negative (wake) and false from true positive (sleep) actigraphic sleep classifications. The contribution of skin temperature measurements to improvements in the accuracy of actigraphic sleep estimates depends on the site of measurement and seems, most conveniently, best if measured on the wrist Consequently, full integration of skin temperature measurement with actigraphy in a single enclosure is one feasible and practical embodiment of the invention In an embodiment, the skin temperature may be measured with a temperature sensor at a wrist of the subject's human body and the motor activity is also measured at the wrist by means of a motion detector, the motion detector and temperature sensor being integrated in a single unit that can be worn by the subject on the subject's wrist.

The inventors also found that finger temperature has good discriminative value, that chest temperature had no discriminative value and that temperature gradients do not outperform single-site temperatures in this respect.

Without wishing to be bound by any theory the present inventors hypothesize that during sleep skin temperature is in part dependent on the environmental temperature and in part on the autonomous nervous system. It is known that the major sleep period occurs during the trough of the circadian rhythm of core body temperature and it has been proposed that skin temperature shows a rhythm that is inversely related to the core body temperature rhythm. The increased skin temperature at night would mainly be due to an increase in skin blood flow causing skin warming and a concomitant dissipation of body heat. These proposed mechanisms probably underlie suggestions in the prior art to use skin temperature or core body temperature, or even gradients of core body temperature to skin temperature as indicators of sleep/wake states. Surprisingly, however, the present inventors have now found that skin temperature is a more useful parameter than core body temperature and, in particular, that there is a significant difference in predictive value between distal skin temperature and proximal skin temperature during ‘sleep’.

These, as well as other, aspects of the invention will be described in more detail and illustrated in the detailed description and examples hereinafter.

Second aspect: monitoring alertness.

A method that meets the needs of the second aspect is defined in claim 25, and an arrangement in claim 27.

Such an arrangement can easily be used by a user in a day-to-day environment, i.e., outside a test environment.

In an embodiment, as defined in claim 33, the arrangement for monitoring alertness is combined with the arrangement for monitoring sleep. The latter embodiment is advantageous in the sense that it can distinguish between three different conditions of the human body: being asleep, being awake but non-alert or being alert.

Third aspect: stimulating sleep.

A method that meets the needs of the third aspect is defined by claim 11, and an arrangement by claim 17.

Such an arrangement can easily be used by a user in a day-to-day environment, i.e., outside a test environment.

Fourth aspect: stimulating alertness.

A method that meets the needs of the fourth aspect is defined by claim 29, and an arrangement by claim 31.

Such an arrangement can easily be used by a user in a day-to-day environment, i.e., outside a test environment.

In an embodiment, as defined in claim 33, the arrangement for stimulating alertness is combined with the arrangement for stimulating sleep.

### BRIEF DESCRIPTION OF THE DRAWINGS

The invention will be explained in detail with reference to some drawings that are only intended to show embodiments of the invention and not to limit the scope. The scope of the invention is defined in the annexed claims and by its technical equivalents. The drawings show:

FIG. 1a shows a setup of a system arranged to perform a method of monitoring sleep.

FIG. 1b shows an embodiment of a sleep monitoring device.

FIG. 2 shows a schematic setup of a computer arrangement that can be used in the setup of FIG. 1.

FIGS. 3a-3c shows three graphs indicating that the addition of skin temperature to actigraphy results in less false negative and less false positive sleep-wake state estimates.

FIGS. 4a, 4b show embodiments of arrangements that can be used to monitor alertness.

FIGS. 5a, 5b, 5c show plots of the average reaction speed (open circles) and percentage of lapses (closed squares) of test persons over ten deciles of subclavicular, finger and wrist temperature respectively. Vertical error bars indicate the standard error of the mean for the performance measures. Horizontal error bars indicate the standard error of the mean for the variation in the range associated with each of the ten temperature percentiles. Linear regression lines are given.

FIG. 6 shows an arrangement for monitoring and manipulating skin temperature in order to control alertness.

FIG. 7 shows an arrangement for manipulating skin temperature in order to control sleep.

FIG. 8 shows an example of a temperature control unit.

### DETAILED DESCRIPTION OF THE INVENTION

### Monitoring Sleep.

In a first aspect of the invention, a method of monitoring sleep in a subject is disclosed comprising measuring within a prescribed interval skin temperature of at least one distal region of the subject's body, comparing said measured skin temperature with a predetermined temperature threshold, and classifying said subject as being asleep if the skin temperature is above said temperature threshold and as being awake if the skin temperature is below said temperature threshold. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the element is present, unless the context clearly requires that there be one and only one of the elements. The indefinite article “a” or “an” thus usually means “at least one”.

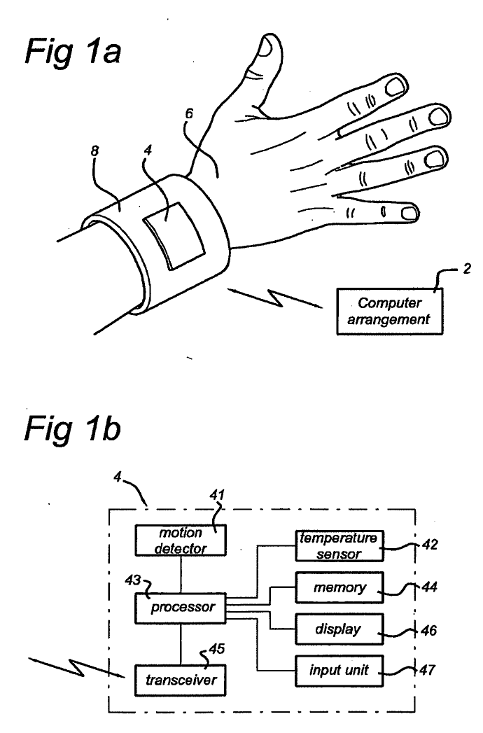

FIG. 1a shows a system that can be used to monitor sleep/wake of a person in accordance with the invention.

The system comprises a computer arrangement 2 that may be a personal computer (PC). The computer arrangement 2 is arranged to communicate with other devices, e.g. via a wireless connection. The system comprises a sleep monitoring device 4, which is shown to be fixed to a band 8 that can be fixed to a humans wrist 6 in any manner known to persons skilled in the art (e.g. by a buckle or by Velcro). However, the sleep monitoring device 4 may be fixed to any other type of holding device arranged to fix the sleep monitoring device 4 to a predetermined part of the human body, Examples of such other body parts will become apparent from the description below.

FIG. 1b shows an example of a sleep monitoring device 4. In the shown embodiment, the sleep monitoring device 4 comprises a processor 43. Moreover, the sleep monitoring device 4 comprises a motion detector 41, a temperature sensor 42, a memory 44, a transceiver 45, a display 46 and an input unit 47 which are all connected to the processor 43.

The motion detector 41 detects any motion of the body part to which the sleep monitoring device 4 is fixed, i.e., in the example of FIG. 1a, the wrist 6. The motion detector 41 outputs motion data to the processor 43 which stores the motion data in memory 44. If required, this motion data is stored inclusive of meta data like the time the motion data was collected, data indicating the user of the sleep monitoring device 4, a serial number of the sleep monitoring device 4, etc.

The temperature sensor 42 is arranged to detect the temperature of the body part to which the temperature sensor 42 is fixed. In the example of FIG. 1a, this is the wrist 6. The temperature sensor 42 outputs temperature data to the processor 43 which stores the temperature data in memory 44.

The memory 44 may be any known suitable type but preferably includes EPROM (electrically erasable read only memory) to permanently store data written into memory 44 by processor 43. It stores a suitable software program arranged to allow the processor 43 to perform the method of the invention.

The transceiver 45 is arranged to wirelessly transmit and/or receive data upon being instructed to do so by processor 43. Data communication may, for instance, be with computer arrangement 2.

The display 46 is arranged to display data as received from processor 43, for instance relating to a current condition of the user, i.e., whether he is asleep or awake. Moreover, the display may show data to the user helping him to set the sleeping monitoring device when the device has to operate.

The input unit 47 is arranged to receive instructions from the user. It may be implemented as a keyboard, a touch screen, or any other suitable input device known to persons skilled in the art,

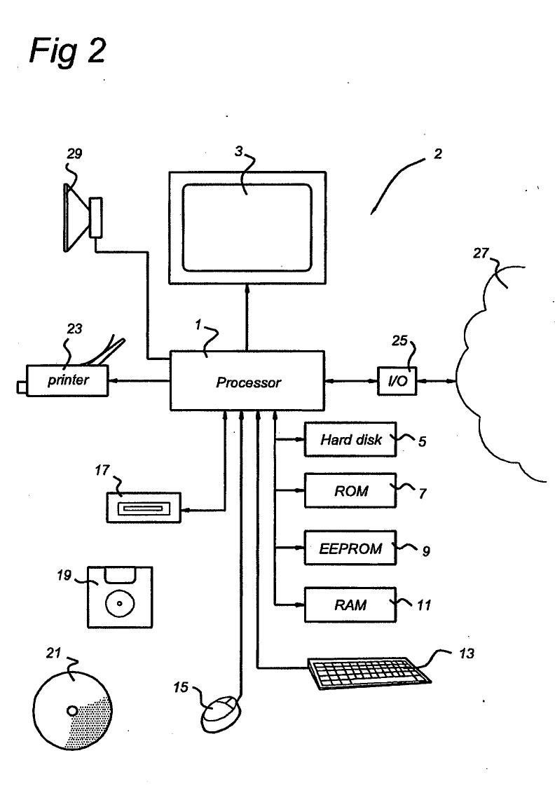

The system may comprise one or more further temperature sensors, or any other sensor to measure physiological or environmental features, for example EEC, EMG, EOG sensor, breathing or heart beat sensors. These further sensors are optional. If present, they provide output data to computer arrangement 2. The further temperature sensors provide temperature data of one or more further body parts. In FIG. 2, an overview is given of a computer arrangement 2 that can be used to carry out one or more tasks in accordance with the invention. The computer arrangement 2 can, for instance, be used to process data of-line and register sleep monitoring data for later use.

The computer arrangement 2 comprises a processor 1 for carrying out arithmetic operations. The processor 1 is connected to a plurality of memory components, including a hard disk 5, Read Only Memory (ROM) 7, Electrically Erasable Programmable Read Only Memory (EEPROM) 9, and Random Access Memory (RAM) 11. Not all of these memory types need necessarily be provided. Moreover, these memory components need not be located physically close to the processor 1 but may be located remote from the processor 1.

The processor 1 is also connected to units for inputting instructions, data etc. by a user, like a keyboard 13, and a mouse 15. Other input units, such as a touch screen, a track ball and/or a voice converter, known to persons skilled in the art may be provided too.

A reading unit 17 connected to the processor 1 is provided. The reading unit 17 is arranged to read data from and possibly write data on a data carrier like a floppy disk 19 or a CDROM 21. Other data carriers may be tapes, DVD, Blu Ray disks, memory sticks, etc. as is known to persons skilled in the art. The data carrier may be provided with a computer program product comprising instructions and data arranged to be read by the processor 1 and, after being read, allowing the processor 1 to perform one or more actions in accordance with the invention.

Such a computer program product may then be loaded in one of the memory components 5, 7, 9, 11. However, such computer program product may, alternatively, be carried by a suitable signal that can be downloaded via a telecommunication network 27.

The processor 1 may be connected to a printer 23 for printing output data on paper, as well as to a display 3, for instance, a monitor or LCD (Liquid Crystal Display) screen, a plasma display panel, or any other type of display known to persons skilled in the art.

The processor may be connected to a loudspeaker 29 to provide an audio signal.

The processor 1 may be connected to communication network 27, for instance, the Public Switched Telephone Network (PST), a Local Area Network (LAN), a Wide Area Network (WAN), the Internet, etc. by means of an I/O unit 25. The processor 1 may be arranged to communicate with other communication arrangements through the network 27.

The processor 1 may be implemented as stand alone system, or as a plurality of parallel operating processors each arranged to carry out subtasks of a larger computer program, or as one or more main processors with several sub-processors. Parts of the functionality of the invention may even be carried out by remote processors communicating with processor 1 through the network 27.

The invention concerns “monitoring sleep in a subject”, meaning, in an embodiment, that periodic sleep or wake classifications are established while the subject is at rest and/or has gone to sleep, in particular, but necessarily, during the night while lying down, e.g. in bed. In an embodiment, the method comprises storing skin temperature and motor activity data and/or the sleep/wake classification data obtained in memory 44, such that it can be analyzed by processor 43 when it runs the program stored in memory 44, in particular to assess the quality and/or quantity of sleep by the subject during a period of data collection. In another embodiment of the invention, skin temperature and motor activity data and/or the sleep/wake classification data collected in accordance with the invention is sent to computer arrangement 2 and stored in one of the memories 5, 7, 9, 11 such that it can be analyzed afterwards.

Sleep is the natural state of bodily rest. Eye movement during sleep is used to divide sleep into two broad types, i.e. rapid eye movement (REM) and non-rapid eye movement (NREM) sleep. Each type has a distinct set of associated physiological, neurological and psychological features. Sleep proceeds in cycles of REM and the four stages of NREM, the order normally being stages 1>2>3>4>3>2> REM. In humans this cycle is on average 90 to 110 minutes, with a greater amount of stages 3 and 4 early in the night and more REM later in the night. Each phase may have a distinct physiological function. Stages 1 and 2 are considered ‘light sleep’, and 3 and 4 ‘deep sleep’ or slow-wave sleep, SWS. They are differentiated solely using EEG, unlike REM sleep which is characterized by rapid eye movements and relative absence of muscle tone. In non-REM sleep there are often limb movements, and parasomnias such as sleepwalking may occur. Sleep timing is controlled by the circadian clock, by homeostasis and, in humans, by willed behavior. The circadian clock, an inner time-keeping, physiology-controlling device, works in tandem with adenosine, a neuro-active compound which inhibits some of the brain processes that are associated with wakefulness. Adenosine accumulates over the course of the day; high levels of adenosine lead to sleepiness. In diurnal animals, sleepiness occurs as the circadian element causes the release of the hormone melatonin and a gradual decrease in core body temperature. It is the circadian rhythm which determines the ideal timing of a correctly structured and restorative sleep episode. Homeostatic sleep propensity, the need for sleep as a function of the amount of time elapsed since the last adequate sleep episode, is also important and must be balanced against the circadian element for satisfactory sleep. Along with corresponding messages from the circadian clock, this tells the body it needs to sleep.

As will be understood, the term wakefulness, as used herein refers to the state of being awake as opposed to being in a state of sleep. The classification that is made based on the data acquired in accordance with the invention at least distinguishes between a state of sleep and a state of wakefulness of the subject. In addition, further classification may distinguish between different stages of sleep.

As noted before, it is known to monitor sleep by continuously measuring a subjects motor activity with an actigraph. An actigraphy is generally used to clinically evaluate insomnia, circadian rhythm sleep disorders, excessive sleepiness, sleep apnea, restless legs syndrome, advanced sleep phase syndrome, delayed sleep phase syndrome, and shift work disorder Additionally, there is some evidence to support the use of an actigraph in the evaluation of patients suspected of jet lag disorder and non-24 hour sleep/wake syndrome. An actigraph is also used in assessing the effectiveness of pharmacologic, behavioral, phototherapeutic or chronotherapeutic treatments for such disorders. The technique is extensively used in academic research and is being increasingly employed in new drug clinical trials where sleep quality is seen as a good indicator of quality of life. An actigraphy is also used by healthy subjects that want to gain insight in their sleeping behavior in order to be able to maintain or establish a healthy lifestyle. As indicated before the present invention involves an improvement of the (sleep) actigraphy methods currently available and it therefore encompasses all the afore-mentioned applications.

Currently available (sleep) actigraphy methods are quite diverse, especially with respect to the (algorithmic) methodologies applied for making sleep/wake classifications based on said data. Actigraphy methods are quite comparable with respect to the part of the body from which motor activity data is collected, which is usually the wrist. Any sleep actigraphy method currently available can be improved by applying the skin temperature data in accordance with the invention, as will be explained further below, and all the sleep actigraphy methods conceivable are therefore encompassed by the present invention.

In currently known (sleep) actigraphy methods, motor activity measurement most often involves detecting acceleration, e.g. using an accelerometer. Single- and multi-axis models are available to detect magnitude and direction of the acceleration.

Because of the wide availability and the ease of implementation it is advantageous for the purposes of this invention that the motion detector 41 is provided with an accelerometer to detect acceleration of the selected body part(s) of the subject. Nevertheless, other methods and devices of detecting motor activity of one or more selected body parts are encompassed as well by the invention, such as using (on-mattress) pressure sensor pad technology, which has been reported to allow detection of gross as well as minute motor activity.

Several methodologies are currently available for making a sleep/wake classification based on the collected motor activity data. The most widely used method of scoring actigraphy data is an algorithm developed by Cole and associates and described in their article entitled “Automatic Sleep/Wake Identification from Wrist Actigraphy” published in Sleep, vol. 15, pp. 461-469 (1992). Successful actigraphy sleep-scoring algorithms such as the Cole et al. algorithm (also known as the Cole-Kripke algorithm) are for use with conventional (number-of-zero-crossings) actigraphs and some algorithms account for the number of counts above a certain threshold. These algorithms are limited to making simple sleep vs. wake distinctions, and cannot distinguish sleep stage changes (e.g., Stage 1 to Stage 2, or Stage 2 to REM) within sleep itself. Other algorithms and methodologies for automated actigraphy scoring have been developed by, for example, Jean-Louis et al., 1996; Sadeh et al., 1989; and Zisapel et al., 1995. Available scoring systems mainly differ along technical aspects, for example, the extent to which activity counts in previous and subsequent epochs influence the scoring of the current epoch, and variation among mathematical principles underlying each scoring system All such known algorithms can be implemented in the computer program stored in memory 44 to allow processor 43 to perform the desired function of monitoring sleep based on output data received from motion detector 41.

As briefly indicated herein before, (sleep) actigraphy may result in one of the following outcomes:
i. false sleep classification (no activity, while the subject is awake);
ii. true sleep classification (no activity and the subject is asleep);
iii. true wake classification (activity and the subject is awake); and
iv. false wake classification (activity, while the subject is asleep).

Outcome i) is a problem, in particular when monitoring sleep in e.g. elderly or insomniacs, which can be awake for a considerable time without exerting any motor activity. Outcome iv) can be a problem, in particular when monitoring subjects that tend to move in their sleep.

### Experiment 1:

The inventors initiated a study in which skin temperature and actigraphy were added to routine polysomnographic recordings. Although recordings are still ongoing, the preliminary results presented here are convincing. In this experiment, the setup of figure 1a was used to which a polysomnograph was added. How such a polysomnograph should be fixed to the human body and how it operates is known to persons skilled in the art and need no detailed explanation here.

### Participants

In the experiment, seventeen participants (9 female, 8 male) with heterogeneous complaints have been recorded and analyzed. Complaints/diagnoses included CR=circadian rhythm disturbance, CFS=chronic fatigue syndrome, AH=Apnea-Hypopnea, PLM-RL=periodic limb movements-restless legs, EDS=excessive daytime sleepiness, DIMS=disturbance of initiating and maintaining sleep, SSM=sleep state misperception, and MS=multiple sclerosis.

### Polysomnography Acquisition

In the experiment, polysomnography (PSG) was obtained using the ambulatory Embla A10 system (“Embla PSG recorder”, Flaga, Reykjavic, Iceland) and included 6 channel EEG, submental EMG, EOG, flow, respiratory effort and tibialis EMG. Recordings were scored at 30-second epoch intervals by experienced sleep technicians and an expert clinical neurophysiologist according to the Rechtschaffen and Kales criteria, using the Somnologica software (Flaga, Reykjavic, Iceland).

### Temperature Acquisition

In the experiment, temperature was assessed with several temperature sensors using iButtons (type DS1922L, MAXIM/Dallas), set to acquire temperature samples at 30 second intervals with a resolution of 0.0625° C. Three different locations were selected to measure skin temperature with such iButtons: for distal skin temperature sites the dorsal side of the base of the middle finger was used, as well as the dorsal side of the wrist, whereas the subclavicular area was used for proximal skin temperature. A fourth iButton was also attached at wrist height, but with its contact surface facing away from the skin in order to assess local environmental temperature. The iButtons were affixed on the side of the non-dominant hand in order to minimize discomfort during the task. The iButtons were started from the same computer that was used to initiate the Embla PSG recorder to ensure adequate synchronization.

### Activity Acquisition

In the experiment, wrist activity was assessed with the Actiwatch AWL actigraph (Cambridge Neurotechnology, Cambridge, UK), set to acquire and store activity at 30 second intervals, An additional Actiwatch Neurologica was worn as well. The Actiwatch was started from the same computer that was used to initiate the Embla PSG recorder to ensure adequate synchronization.

Data analysis

The 30 second epochs of PSG sleep stages, temperature and activity were synchronized in single files for each participant. In order to promote a conservative estimate of the usefulness of co-registration of temperature with activity, only the period between lights out and final awakening was analyzed. The match between PSG determined wakefulness and actigraphic activity was subsequently classified for each epoch as:

(1) false negative: no activity yet wakefulness according to the PSG (major problem of actigraphy);

(2) true negative: no activity in agreement with PSG sleep

(3) true positive: activity in agreement with PSG wakefulness

(4) false positive: activity yet sleep according to the PSG (secondary problem of actigraphy).

For each of the four classes, the average skin temperatures on the indicated locations were calculated.

### Results

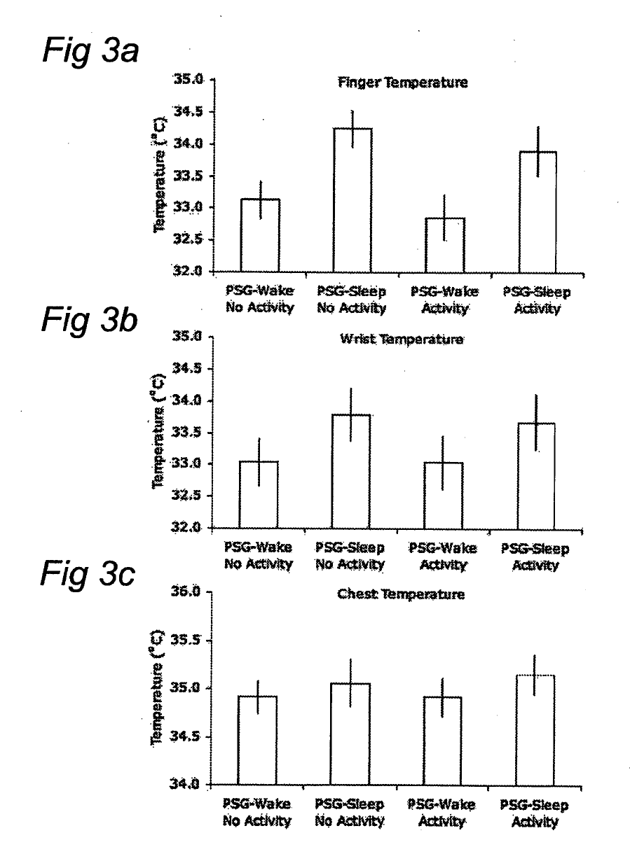

FIGS. 3a-3c shows test results for the seventeen participants, i.e., as averages for all participants. Measurements made at the finger are shown in the top figure, at the wrist in the middle figure and at the chest in the bottom figure. They show temperature over epochs where actigraphic activity classified the PSG-verified wakefulness as, from left to right, false and true negative, true and false positive.

As FIGS. 3a-3c shows, there is an extremely promising difference in the temperatures that characterize the four classifications. The tests show that finger temperature discriminates false from true negative (p=0.002) and false from true positive (p=0.004) classifications. Also wrist temperature discriminates false from true negative (p=0.0005) and false from true positive (p=0.007) classifications. Chest temperature has no discriminative value; neither do gradients outperform single-site temperatures.

Measuring a temperature at a distal portion of the body, like at a finger or at a wrist seems to be a reliable indicator of whether or not the person concerned is sleeping or awake. In the present sample of participants, measured under normal circumstances at home, if the temperature at such a distal portion is, say, below 33.5° C., the person concerned is most probably awake. If, however, the person's temperature at the distal portion is, say, above 33.5° C. and immobile then the person concerned is most probably sleeping. Both the most optimal measuring site and the threshold temperature may be optimized for specific individual and environmental characteristics including age, diagnosis, gender and environmental temperature. The optimization may involve a single site or multiple sites.

As FIGS. 3a-3c also shows, there is a significant difference between the average temperature at a wake condition and a sleep condition. The figures show that this difference may be in the order of more than 1.0° C. when measured at the finger and more than 0.9° C. when measured at the wrist. This measure can also be used by the sleep monitoring device 4 to establish whether a person is asleep or awake. To do so, the sleep monitoring device 4 is programmed to monitor a distal temperature of the body over time such that it can establish whether such a temperature change occurs. The threshold of temperature change indicating a change from one condition to the other may differ per person. So, in an embodiment, this threshold is set in the sleep monitoring device 4 in dependence on the person. Both the most optimal measuring site and the threshold temperature may be optimized for specific individual and environmental characteristics including age, diagnosis, gender and environmental temperature. The optimization may involve a single site or multiple sites.

Moreover, the rate of change of the measured temperature at the distal body part is an 5 indicator of whether the person is falling asleep from a condition of being awake or whether the person is waking up from a condition of being asleep. The measured rate of change of the temperature can be compared by the sleep monitoring device 4 against a threshold value which may be different per person.

### Conclusion

An algorithm based on skin temperature measured at distal parts of the body can be applied to establish whether a person is asleep or awake. Moreover, such an algorithm can be used to improve the discrimination between false positive and true positive actigraphic estimates of both sleep and wakefulness.

To that end, in the embodiment of figure 1a of the invention, the processor 43, when running the program stored in memory 44 performs basically the following actions:

a. receiving motion data from motion detector 41;

b. receiving temperature data from temperature sensor 42;

c. establishing whether the person, who is sensed by the motion detector 41 and temperature sensor 42, is asleep or awake in dependence on the motion data as verified by the temperature data where the temperature data is compared against a threshold temperature, where a temperature above said temperature threshold is an indication that the person is most probably asleep and below said threshold is an indication that the person is most probably awake. As an alternative or additional measure the change of temperature may be used as an indication of the bodies sleep condition.

The temperature threshold will be dependent on the selected distal portion of the person's body but may be in the range between 33.3 to 33.7° C.

The embodiment shown in figure 1a is very advantageous since it shows a sleep monitoring device 4 which integrates both motion detector 41 and temperature sensor 42 in one single device that can be worn on the wrist whereas FIGS. 3a-3c show that the wrist is a perfect location for both collecting motion data and temperature data.

In an alternative embodiment, the temperature threshold is person specific, In that case, the computer arrangement 2 may be arranged to receive a temperature threshold setting signal from a user of the system. The user fixes the temperature sensor 42 to his body, after which he inputs the temperature threshold setting signal to the sleep monitoring device 4 via input unit 47. The processor 43 receives both the temperature data from the temperature sensor 42 and the temperature threshold setting signal from the user and stores the received temperature data as a temperature threshold in memory 44.

Alternatively, auto-calibration in order to determine the individual optimal threshold may be applied. One implementation of this would use one or multiple sleeping periods

(1) to determine the 95% confidence interval of skin temperatures during the last part of the, say 5%, longest immobile periods during the sleep period, which are highly likely associated with deep sleep;

(2) subsequently, to determine the 95% confidence interval of skin temperatures during the, say 5%, longest continuous activity periods during the sleep period, which are highly likely associated with a complete absence of sleep;

(3) finally, to set the threshold such that it maximizes the distance between the two defined confidence intervals.

As explained herein before, it was found that the skin temperature of distal regions rather than that of proximal regions of the body is suitable for discriminating between false and true sleep classifications based on motor activity. In accordance with this embodiment of the present invention distal regions of the body include any portion of the head, arms and legs, including hands and feet. Preferably the distal region is selected from regions of an elbow, a lower arm, a wrist, a hand, a finger, a lower leg, an ankle, a foot and toe of the subject, more preferably from regions of a lower arm, wrist, hand, finger, lower leg, ankle, foot and toe, most preferably from regions from the wrist.

Skin temperature sensors may be integrated in miniature recorders, either stand alone or within the sleep monitoring device 4, and e.g. strapped on the wrist as a wristwatch or strapped or taped to any other location on the body.

As noted before, the method comprises measuring skin temperature of at least one distal region. As will be understood, skin temperature measurements may be taken and used in accordance with the invention from one individual location as well as from several locations including proximal locations. Although it was found that the use of skin temperature gradients from one region of the body to another does not outperform the use of data obtained from a single location, the use of gradients is not excluded from the scope of the invention.

The term “prescribed interval” as used herein, indicates that motor activity based sleep/wake classifications from one time interval and its surrounding time intervals are verified on the basis of skin temperature measurement data obtained in the same time interval and possibly their surrounding intervals as well. That term is not meant to impose any specific limitation with regard to the number of measurements taken in each ‘interval’ or with regard to the actual length of the interval. As will be understood by the skilled person it is advantageous to make sleep/wake classification during several intervals in one episode of sleep or going to bed. Consecutive ‘intervals’ may be selected as non-overlapping intervals. But it is equally possible that intervals overlap or that the intervals alternate with periods during which no measurements are taken. Typically, a sleep/wake classification is made every 20, 30 or 60 seconds, preferably every 30 seconds.

Furthermore, the method may encompass detecting every movement of the selected body part(s) of the subject by motion detector 41, i.e. that motor activity is monitored continuously, while skin temperature measurements may be taken with temperature sensor 42 continuously or periodically, preferably periodically, more preferably once every 20, 30 or 60 seconds, preferably once every 30 seconds.

The reliability of the sleep/wake classifications obtained in accordance with the present invention may be further improved by measuring one or more additional physiological parameters that alter in going from a state of sleep to a state of wakefulness or vice versa. Such parameters, for example, include eye movement as measured by an eye movement sensor (not shown), core body temperature as measured by one or more further temperature sensors, heart rate as measured by a heart rate sensor (not shown), muscle tone, etc. Such body core temperature sensors may be implemented as a temperature measurement grid integrated in a bed. Such additional measurements may be included in the present method without departing from the scope of the invention, as will be understood by a person skilled in the art.

### Monitoring Alertness.

A second aspect of the present invention relates to alertness. The present inventors have found that proximal skin temperature measurements can be applied to obtain an estimate of the current and near future alertness level of a subject.

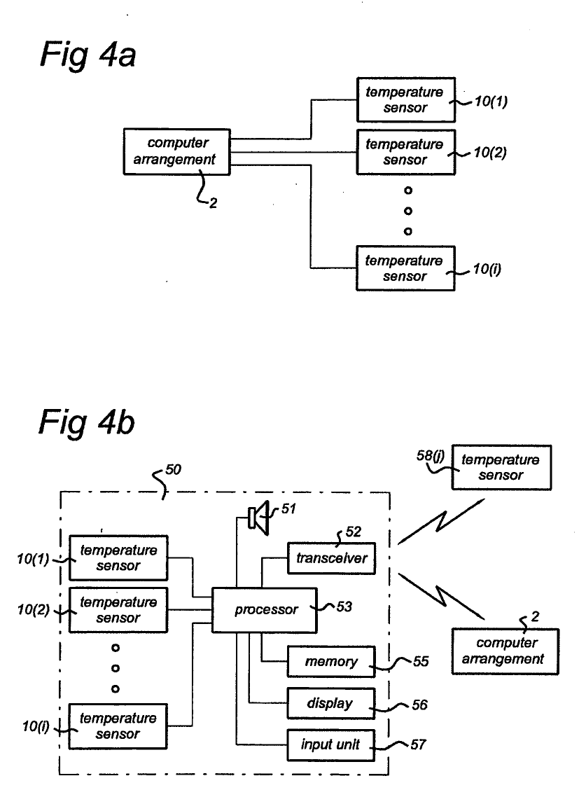

Monitoring alertness can be established by a alertness monitoring setup as shown in FIG. 4a. The figure shows computer arrangement 2 connected to one or more temperature sensors 10(i), i=1, 2, . . . , I, arranged to be attached to the skin of a human body. The connections may be wired or wireless. The computer arrangement 2 may be the same one as shown in FIG. 2. Memory 5, 7, 9, 11 stores a suitable computer program to allow processor 1 to monitor alertness of a person to whom the one or more temperature sensors 10(i) are attached. If processor 1, when it runs the computer program, establishes that the person is not alert anymore it can generate a control signal that may be sent to a warning system or stimulation device, for example loudspeaker 29 which then produces a warning signal to the user.

In one embodiment, the alertness monitoring setup comprises an integrated alertness monitoring device distinct from the computer arrangement 2. This embodiment is shown in FIG. 4b. Such a alertness monitoring device can be integrated in a single enclosure which is worn on the body.

FIG. 4b shows a alertness monitoring device 50 with a processor 53. The processor 53 is connected to a loudspeaker 51, a transceiver 52, a memory 55, one or more temperature sensors 10(i), a display 56 and an input unit 57. There may also be one more external temperature sensors 58(j) (j=1, 2, . . . , J).

The temperature sensors 10(i) are arranged to detect the temperature of the body part to which the temperature sensors 10(i) are fixed. The temperature sensors 10(i) output temperature data to the processor 53 which stores the temperature data in memory 55.

The one or more external temperature sensors 58(j) are, for instance, arranged to measure the environmental temperature and send environmental temperature data to the alertness monitoring device 50.

The memory 55 may be any known suitable type but preferably includes EPROM (electrically erasable read only memory) to permanently store data written into memory 55 by processor 53. It stores a suitable software program arranged to allow the processor 53 to perform the method of the invention.

The transceiver 52 is arranged to wirelessly transmit and/or receive data upon being instructed to do so by processor 53. Data communication may, for instance, be with computer arrangement 2 and the one or more external temperature sensors 58(j).

The display 56 is arranged to display data as received from processor 53, for instance relating to a current condition of the user, i.e., whether he is alert or non-alert. Moreover, the display 56 may show data to the user helping him to set the alertness monitoring device 50 when the device has to operate.

The input unit 57 is arranged to receive instructions from the user. It may be implemented as a keyboard, a touch screen, or any other suitable input device known to persons skilled in the art.

### Experiment 2:

While using the setup as shown in FIG. 4a, the inventors performed a study in which skin temperature was measured with temperature sensors 10(i) during repeated performance on an alertness task where lapses of alertness and reaction times were continuously monitored by computer arrangement 2 during sustained requirements.

### Participants

Eight healthy participants (5 males, 22-47 years of age, mean±SD: 30.1±8.1 years) participated in the protocol. All but one subject were right-handed, and none of the subjects had any known history of sleep-related disorders. Subjects were instructed to keep the amount and time of caffeine intake equal on the two days.

Block design

The protocol consisted of 4 blocks a day for two days. During each block, subjects were asked to sit in a dimly-lit room (±15 lux) at room temperature. To assess skin temperature, temperature sensors 10(i) in the form of thermologgers were attached to different portions of the subject's body, who was then allowed to acclimatize to the conditions by reading for 10 minutes. After this acclimatization period, processor 1 presented a computerized task battery to the subjects via monitor 3. In this task battery, subjective sleep quality of the previous night, as well as daytime alertness was assessed via 100 mm visual analog scales. Sleepiness was assessed by using the Karolinska Sleepiness Scale (KSS). Each block lasted for approximately 40 minutes in total, and depending on availability of the subject, the first block started between 09.00 and 11.00 am. The interval between two consecutive blocks was held constant at 2 hours.

### PVT

The task battery included an adapted “psychomotor vigilance task” (PVT), wherein processor 1 instructed subjects to respond to a stimulus which was very briefly displayed on monitor 3. Processor 1 displayed a black fixation cross against a gray background on monitor 3 for a random interval (4-14 seconds, 84 ms resolution). After this variable fixation period, processor 1 flashed a hyphen (-) on monitor 3 for 25 ms, after which the black fixation cross returned and the participant was allowed to respond within 1 second at most. Processor 1 instructed the subjects to respond by pressing a button (e.g., one as available on mouse 15) with the index finger of their dominant hand as soon they saw the fixation cross change. Omissions and response durations longer than 500 msec were regarded as lapses. In order to obtain a normal distribution, the inverse of the observed reaction times was taken and denoted as ‘speed’.

### Temperature Acquisition

Temperature sensors 10(i) to assess the skin temperature were iButtons (type DS1922L, MAXIM/Dallas), which were set to acquire temperature samples at 30 second intervals with a resolution of 0.0625° C. Three different locations were selected to measure skin temperature: for distal skin temperature sites the dorsal side of the base of the middle finger was used, as well as the dorsal side of the wrist, whereas the subclavicular area was used for proximal skin temperature. A fourth iButton was also attached at wrist height, but with its contact surface facing away from the skin in order to assess local environmental temperature. The iButtons were affixed on the side of the non-dominant hand in order to minimize discomfort during the task.

Apart from the temperature as a parameter other parameters may be applied: it is conceivable that the optimally discriminative site (for alertness drop detection) varies with body position (supine, sitting, standing), age, gender, body weight, sleep history, time awake, time of day, individual circadian phase, environmental temperature and humidity, environmental light and their interactions. Such parameters can be stored in the memory in the alertness monitoring setup and used in the calculation to establish whether a person is alert or not. Some of them can be measured in real-time by suitable sensors, like body position, environmental temperature, humidity and environmental light.

Data analysis

It turned out that temperature ranges differed between subjects. The computer arrangement 2 was programmed to produce a graphical representation of the relation between performance and temperature, where the temperature range of each individual subject was subdivided into ten deciles, and the average speed and percentage of lapses was calculated within each decile,

In order to test for the statistical significance of the relations, computer arrangement 2 applied a mixed effect regression analysis to account for a nested data structure of responses nested within four blocks nested within two days, again nested within eight subjects. Computer arrangement 2 made an analyses using a software package known as “MLwiN” (a software package for fitting multilevel models, available from Centre for Multilevel Modelling, University of Bristol). stored in memory 5, 7, 9, 11.

### Results

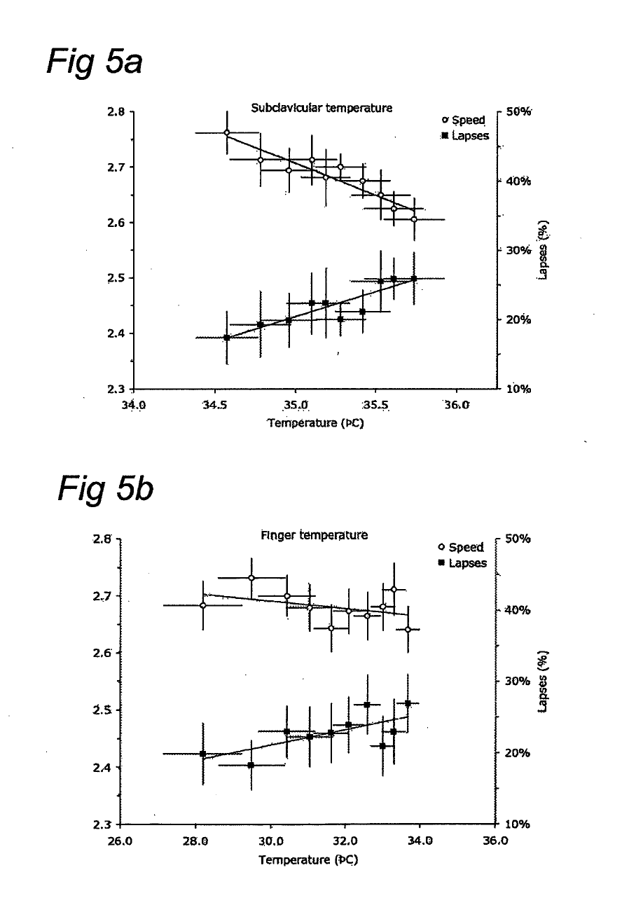

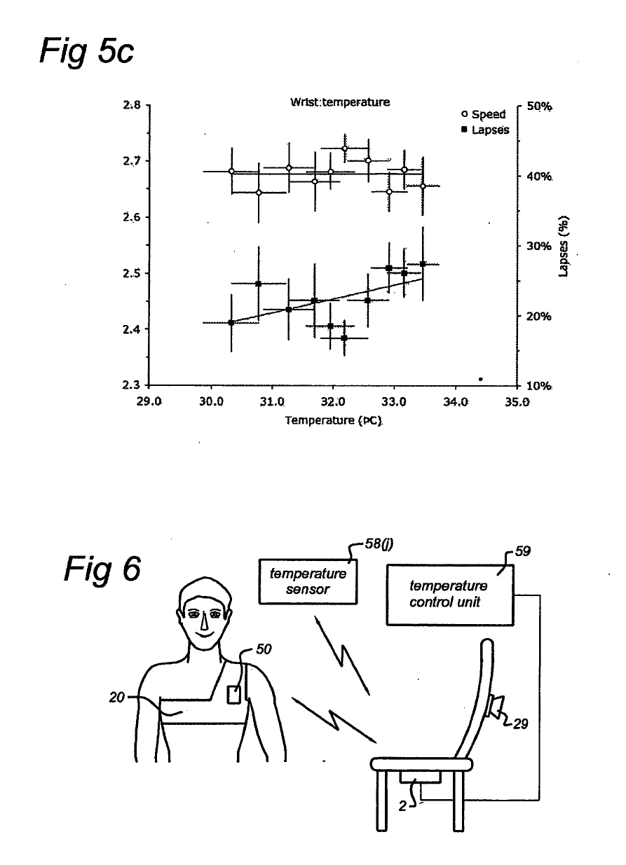

FIGS. 5a, 5b, 5c show plots of the average reaction speed (open circles) and percentage of lapses (closed squares) of test persons over ten deciles of subclavicular, finger and wrist temperature respectively, as generated by computer arrangement 2. Vertical error bars indicate the standard error of the mean for the performance measures. Horizontal error bars indicate the standard error of the mean for the variation in the range associated with each of the ten temperature percentiles. Linear regression lines are given in each one of these FIGS. 5a, 5b, 5c.

FIGS. 5a, 5b and 5c show that an increase in subclavicular temperature predicts a decrease in performance of the user, less so for finger temperature and marginally so for wrist temperature. Mixed effect linear regression analysis indicates a strong negative predictive value of the proximal (subclavicular) skin temperature decile (p=0.00001) for the speed of the performance of the user. Likewise both proximal (p=0.003) and, less so, wrist (p=0.03) temperature deciles have a positive predictive value for the percentage of lapses.

Subsequent analyses indicates that gradients (finger to wrist, wrist to subclavicular and finger to subclavicular) yields no better predictive value than the individual locations provided.

### Conclusion

Proximal temperature measurements may contribute significantly to an estimate of momentary alertness. The decile ratings account for 23% of the variance in response speed and 11% of the variance in lapse rate. This important reduction of 23% in the uncertainty about the response speed and 11% in the uncertainty about lapse rate may be further improved by combining chest temperature measurement with other indicators of alertness that may be derived from the central nervous system, autonomous nervous system or overt behavior.

As already indicated before, skin temperature is in part dependent on the environmental temperature and in part on the autonomous nervous system, which regulates the flow of warm blood through the skin. As supported by the above experiment, in a stable thermal environment measuring variations in the skin temperature provides insight in the state of the autonomous nervous system. The state of the autonomous nervous system in turn provides information on the level of alertness. The present inventors were not only able to demonstrate that the skin temperature can be measured to obtain an estimate of the current and near future alertness of the alertness level, but also discovered that the proximal skin temperature of the body, and more specifically the trunk, provides more reliable estimates than that of other regions.

Hence in a further aspect of the invention, a method of monitoring a subject's level of alertness is provided comprising periodically or continuously measuring skin temperature of the trunk of said subject's body by means of at least one temperature sensor 10(i) and determining the subject's level of alertness on the basis of said skin temperature by said computer arrangement 2, wherein a skin temperature above a predetermined threshold is indicative of a too low alertness level. Such temperature threshold is area and person specific and may, for instance, be about 35° C. when the temperature is measured at the subclavicular area. Moreover, the method may involve monitoring a temperature change of the trunk area over time and determine the subject's level of alertness based on the measured temperature difference within a certain time frame.

Monitoring of alertness may be of particular interest in subjects carrying out certain tasks that require a high amount of mental effort and organization, while such tasks involve certain risks, such as driving a vehicle or operating (heavy) machinery, but also performing purely mental or intellectual tasks. Alertness is related to psychology as well as physiology. People who (structurally) lack alertness may have narcolepsy, attention deficit disorder, chronic fatigue syndrome, depression, Addison's disease, or sleep deprivation.

The invention is not restricted to the trunk area. In an embodiment, for use in a car, distal body temperature, e.g. at fingers or other parts of a hand, can be measured by integrating the temperature sensors 10(i) in a steering wheel. Such temperature sensors 10(i) may be either pressure sensitive or environmental light-sensitive sensors located at a high density in the steering wheel. Only a few temperature sensors 10(i) at a time will have good contact with the skin of the hands, and it are only these temperature sensors 10(i) that should contribute at any specific time to the momentary skin temperature estimate made by processor 1. Selection can be based on those sites with high pressure or low environmental light reception.

Several factors may be used as parameter for adaptive modification of thresholds of trunk skin temperature and optimal sites of measurement for establishing whether a person is alert or not. It is conceivable that the optimally discriminative site (for alertness drop detection) varies with, for example, body position (supine, sitting, standing), physical activity history, age, gender, body weight, sleep history, time awake, time of day, individual circadian phase, environmental temperature and humidity, environmental light and their interactions. Such parameters can be stored in the memory in the alertness monitoring setup and used in the calculation to establish whether a person is alert or not. Some of them can be measured in real-time by suitable sensors, like body position, physical activity, environmental temperature, humidity and environmental light.

In an embodiment of this aspect of the invention, the method comprises determining changes in skin temperature of the selected region(s) of the subject's body to determine whether alertness is increasing or decreasing. More in particular, the method comprises periodically or continuously measuring the skin temperature of the selected region(s) of the subject's body, wherein an increase in temperature during a certain interval is indicative of a decrease in alertness during said interval,

It is within the skills of trained professionals to provide suitable methods of measuring skin temperature of a selected region of a subject's body and the present invention is not limited to any particular method. Nevertheless, in accordance with a suitable embodiment of the invention chest or subclavicular skin temperature data is acquired by a temperature sensor called “Vitalsense Dermal Patch” that transmits wirelessly and is available from Respironics.

As stated before, the present method comprises periodic or continuous measurement of the skin temperature of the selected region(s) of the subject's body. In accordance with a suitable embodiment of the invention measurements are taken periodically. In such a method the periods or intervals between subsequent measurements may be equal in length.

The present method and arrangement can be used for monitoring alertness in any subject. In an embodiment of the invention said subject is a subject performing any kind of task for which a certain minimum level of alertness is required, especially a subject operating a vehicle or heavy machinery, or performing monitoring task requiring high alertness. In an embodiment of the invention, said subject performing said task is at risk of being insufficiently alert, more in particular said subject may be suffering from sleep deprivation, narcolepsy, attention deficit disorder, chronic fatigue syndrome, depression and/or Addison's disease, or medication-related drowsiness or sleepiness.

An arrangement for such a method is shown in FIG. 6. It may comprise a binding device for binding the alertness monitoring device 50 to the required portion of the human body. Such a binding device may be an article of clothing comprising the alertness monitoring device 50, e.g., a ribbon or tape 20 that can be worn on the chest of the body such that, in use, the temperature sensor(s) 10(i) inside alertness monitoring device 50 is (are) pressed against the subclavicular area to sense the skin temperature in that area. The binding device may have any other suitable form to bind the temperature sensor 10(i) to the selected location on the body.

In an embodiment, also shown in FIG. 6, the invention relates to a chair 22 in which the computer arrangement 2 is integrated. In such an embodiment, the computer arrangement 2 may only comprise processor 1, one or more of the memories 5, 7, 9, 11 storing a suitable computer program and a warning system or stimulation device, for example loudspeaker 29 to generate a warning signal to the user. The computer arrangement is connected to the temperature sensor 10(i) accommodated in the binding device. Once the processor 1, based on the temperature signal received from temperature sensor 10(i), establishes that the user is not alert anymore in accordance with the method explained above it generates a warning signal to be transmitted by the loudspeaker 29 to warn the user.

In an embodiment, the temperature sensors 10(i) are implemented as microsensors with integrated temperature recording and wireless signal transmission properties that can be attached to the skin of the user with adhesive tape or bands to the desired location of the trunk. The transmitted signals can be received and processed by the computer arrangement 2.

In another embodiment, the temperature sensors 10(i) are no part of alertness monitoring device 50 but are implemented as a grid of miniature temperature sensors that can be integrated in the chair 22 beneath and/or at the back of the user. As a further alternative, such a grid of miniature temperature sensors can be integrated in a safety belt as worn in a car.

During normal use of the chair 22, only a restricted selection of all temperature sensors 10(i) will be close enough to the skin of the user to provide a reliable estimate of skin temperature. Two methods may be applied alone or in parallel to aid automated selection of these temperature sensors 10(i). First, the temperature sensors 10(i) measuring the highest temperature are the temperature sensors 10(i) most likely to be in good contact with the skin, and their output data will be selected by computer arrangement 2 to establish whether the user is alert or not. Second, a grid of pressure sensitive sensors may be integrated with the grid of temperature sensors 10(i), At those sites where the pressure is highest, close contact with the skin is most likely. The output from temperature sensors 10(i) at those locations will be selected by computer arrangement 2 to establish whether the user is alert or not.

### Manipulating Sleep.

The present inventors have also found that temperature manipulations of the skin may be suitable for promoting sleep in a subject. Promoting sleep may be desirable in subjects suffering from sleep disorders such as insomnia and narcolepsy, and from poor sleep in association with e.g. ADHD, depression, aging and dementia and/or in subjects that are required to sleep at times of the day when sleep is not favored by the endogenous circadian rhythm e.g. in case of shift-work or jet-lag. The inventors have discovered that it is possible to promote sleep by subtle manipulation of the skin temperature. The mechanism underlying these findings are unknown. Without wishing to be bound by any particular theory, it is hypothesized that brain areas involved in sleep regulation are somehow sensitive to skin temperature. The promoting of sleep is brought about, in accordance with the present invention, by subtle temperature manipulations in specific temperature ranges, which may vary between subjects; the simple use of heating blankets for example tends to disturb rather than to promote sleep. The efficacy of sleep promotion through skin temperature manipulations in accordance with the invention is furthermore believed to be strongly dependent on the specific site of the skin where temperature is manipulated.

The present inventors have performed a study to identify suitable locations, as reported in an article published in 2008: Raymann et al., Skin deep: enhanced sleep depth by cutaneous temperature manipulation, Brain (2008), 131, 500-513, the entire content of which is incorporated herein by reference. In said article the effects of subtle skin temperature manipulations on sleep are described. According to Raymann et al., at least in an environment where the environmental temperature was kept substantially constant at 21° C., distal skin warming enhances REM sleep and suppresses light sleep in young and older subjects without sleep complaints whereas proximal warming results in deeper nonREM slow wave sleep and suppressed wakefulness. In elderly insomniacs proximal warming enhances nonREM slow wave sleep and REM sleep whereas distal warming enhances slow wave sleep and suppresses REM sleep. The results also show that mild skin temperature manipulations can be chosen such as to significantly reduce early morning awakening and enhance deeper sleep stages. No changes in core body temperature were observed when manipulating skin temperature, such that core body temperature cannot have mediated any of the effects. So, the experiment showed that sleep can best be manipulated by controlling at least one of a proximal and distal skin area depending on the person concerned and the stage of sleep concerned (deep nonREM slow wave sleep, REM, etc.). Other factors that may influence sleep are body position (supine, sitting, standing), age, gender, body weight, sleep history, time awake, time of day, individual circadian phase, environmental temperature and humidity, environmental light and their interactions.

Mild skin temperature manipulations can be chosen such as to significantly reduce early morning awakening and enhance deeper sleep stages. Early morning awakening and a lack of deep sleep are typical findings even in elderly people who do not have sleep complaints. Elderly participants show such a pronounced sensitivity to skin temperature manipulations, that the induction of an increase of 0.4° C. in skin temperature typically lowers the probability of being awake at 6:00 in the morning by a factor 14, from 0.58 to 0.04, for elderly without sleep complaints, and by a factor 5, from 0.36 to 0.07, in elderly insomniacs. In addition, subtle skin warming significantly restores the age-related decrease in slow wave sleep, often considered the most physiologically restorative stage of sleep. Typically, the induction of a 0.4° C. increase in skin temperature doubles the overnight occurrence of slow wave sleep from 8 to 14% in elderly without sleep complaints and from 4 to 9% in elderly insomniac. Hence, in an embodiment of the invention the method comprises controlling skin temperature in the subject, preferably an elderly subject with or without insomnia, such as to to clamp the skin temperature 0.2-0.8° C., most preferably 0.3-0.6° C., higher as compared to the skin temperature of the selected portion observed without or prior to manipulation, typically under normal sleeping circumstances.

More specific, it was found that a bed in which a person is sleeping can best be kept at a temperature, on average, above 33.5° C., 33.2° C. and 33.1° C. for young adults, elderly subjects without sleep complaints and elderly people with sleep complaints, respectively. Simply applying a heating blanket turned out to be insufficient: the microclimate temperature in bed should be controlled to prevent the body temperature from getting too high and adversely affect sleep, most likely by activating heat stress responses. Skin temperature is preferably clamped close to an optimum temperature, not much higher or lower, preferably within a 0.4 ° C. range around the optimum, most preferably a 0.2° C. range. The optimum temperature may show variability between people and variability due to factors including age, gender, body weight, sleep history, time awake, time of day, individual circadian phase, environmental temperature and humidity, environmental light and their interactions.

In a particularly preferred embodiment of the invention the skin temperature manipulations do not induce the mean proximal skin temperature to drop below a certain minimum temperature. Preferably said minimum temperature is 34.40° C., more preferably 34.80° C., most preferably 34.85° C.

The temperature of the invention, the skin temperature manipulations are applied while keeping the temperature of the environmental air, typically the air breathed and to which the face is exposed, within a predetermined value. Without wishing to be bound by any theory, it is believed that elevating ambient temperature instead of directly manipulating the proximal and distal skin, will not lead to any comparable sleep improvements, because elevated air temperatures may be experienced as uncomfortable. Thus, in a preferred embodiment the skin temperature manipulations are performed while keeping the temperature of the environmental air within the values normally conceived as most comfortable, typically within the range of 18-21° C. In a preferred embodiment of the invention, a method as defined before is provided, wherein temperature manipulations are limited to the distal and/or proximal skin area.

The magnitude, body location and timing of the skin temperature manipulation influence the effectiveness of the present method in improving sleep. A thermal sleep treatment should preferably aim at individualized and time-of-night-dependent control of skin temperature within predetermined ranges during sleep. Thus, when manipulating sleep, in accordance with the invention, preferably a closed loop system is used. The closed loop system is arranged to control the skin temperature at the predetermined location within a predetermined optimal range. The exact location depends on several factors, as will be explained below.

In order to allow a closed loop temperature control of a selected part of a subject's body in order to promote a state of sleep, the temperature control unit 24, as shown in FIG. 8, comprises one or more temperature sensors 241, and one or more temperature manipulation units, for instance in the form of one or more cooling elements 242 and one or more heating elements 243. All these units are connected to an I/O unit 244. The one or more temperature sensors 241 are arranged to measure skin temperature on the selected part of the subject's body and to send suitable output signals to that effect to computer arrangement 2, e.g., via I/O unit 244. However, alternatively, temperature sensors 241 may be implemented as a plurality of microsensors with integrated temperature recording and wireless signal transmission properties that can be attached to the skin of the user with adhesive tape or bands, e.g. around legs, arms or trunk. The transmitted signals can be received with computer arrangement 2.

The computer arrangement 2 receives the output signals from the one or more temperature sensors 241 and generates the suitable control signal. The one or more temperature manipulation units 242, 243 are arranged to receive the control signal from the computer arrangement 2 and to control the skin temperature of the selected body part based on the received control signal such that skin temperature of the selected body part remains within a predetermined temperature range. Both the selected body part and the temperature range may depend on personal characteristics of the subject concerned and the desired state of sleep to be obtained.

In an embodiment, temperature sensors 241 are used that are implemented as a grid of miniature temperature sensors that are integrated in the bed 26 beneath and/or above the sleeping person.

During normal sleep, only a restricted selection of all temperature sensors 241 will be close enough to the skin of the user to provide a reliable estimate of skin temperature. Two methods may be applied alone or in parallel to aid automated selection of outputs of these temperature sensors 241 by computer arrangement 2. First, the temperature sensors measuring the highest temperature are the temperature sensors most likely to be in good contact with the skin. The output signals of these temperature sensors will be selected by computer arrangement 2 to establish the skin temperature of the selected body part. Second, a grid of pressure sensitive sensors (not shown) may be integrated with the grid of temperature sensors 241. Such pressure sensitive sensors are arranged to send pressure measurement signals to computer arrangement 2 via I/O unit 244. At those sites where the pressure is highest, close contact with the skin is most likely. The output signals of the temperature sensors 241 at these sites will be selected by computer arrangement 2 to establish the skin temperature of the selected body part.

The computer arrangement 2 may be arranged to integrate information of the temperature sensors in a region-specific way (e.g. trunk, legs, arms), to compare this information with default and individualized optimal temperature settings as stored in its memory, and to control the temperature manipulation units 242, 243 accordingly. Individualization of temperature can be done through input by a user or operator to the computer arrangement 2 of subjective sleep quality after waking up in the morning and by integration of sleep monitoring data, including polysomnography and actigraphy data.

Apart from a temperature control unit to manipulate the skin temperature at a predetermined location to promote sleep, a device can be used to monitor whether the subject is really sleeping or not. Therefore, advantageously, such a closed loop system comprises a sleep monitoring device as explained above with reference to FIGS. 1a, 1b to measure a distal skin temperature and, optionally, to perform a motion measurement and to give feedback for the system that has to manipulate the condition of sleep. In addition, other suitable sleep monitoring devices can be used to improve such a feedback.

Thus, in a preferred embodiment of the invention, a sleep manipulating arrangement is provided comprising a sleep monitoring arrangement as described above, with reference to the first aspect of the invention, a computer arrangement and one or more temperature manipulation units, the computer arrangement being arranged to control said skin temperature of said one or more selected portions of said body within a predetermined temperature range associated with a state of sleep of said subject via said one or more temperature manipulation units, based on whether said subject is awake or asleep.

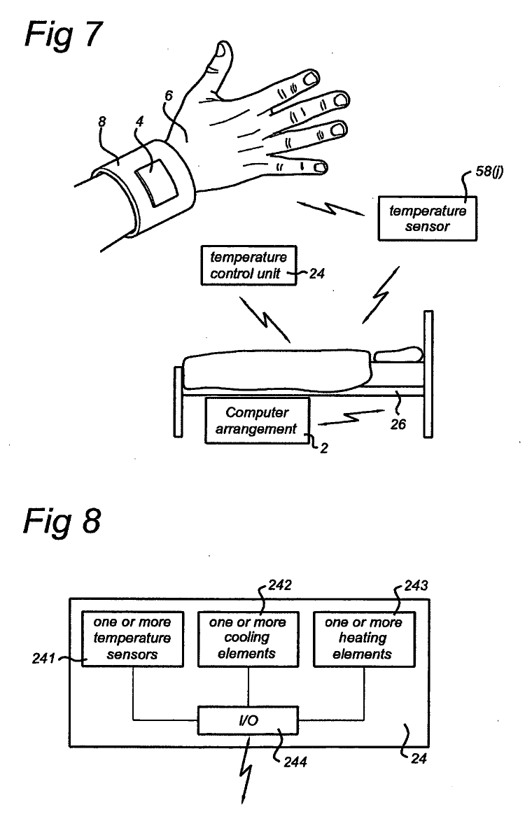

An embodiment of an arrangement for both monitoring and manipulating the bodies temperature in order to manipulate sleep is shown in FIG. 7. The setup as shown comprises a sleep monitoring device 4 similar to the one shown in FIG. 1a. The setup comprises a temperature control unit 24 arranged to manipulate the temperature of a selected portion of the body. The temperature control unit 24 is arranged to communicate with computer arrangement 2, e.g., in a wireless way. The computer arrangement 2 can be implemented in a simple way: it may only comprise processor 1 and one or more of the memories 5, 7, 9, 11 storing a suitable computer program to allow processor 1 to generate a suitable control signal for temperature control unit 24. The setup also shows a bed for a subject to sleep in and one or more temperature sensors 58(j) to measure environmental temperature and to send suitable environmental temperature measurement signals to computer arrangement 2.

The temperature control unit 24 can be implemented in many different ways. For instance, a thermosuit arranged to control skin temperature differently at different locations of the body covered by the thermo suit can be used. However, this may not be a suitable implementation at home. In a suitable implementation for homes, the temperature control unit 24 comprises a grid of actuators for temperature manipulation that can be integrated in the bed 26 beneath and/or above the sleeping person. These can be addressed by the computer arrangement 2 in a region-specific way. Actuators may be implemented either with thermo-electric devices for heating (e.g. heating elements) and cooling (e.g. peltier elements), or by control of valves of a tubing system through which temperature-conditioned gas or liquid can be circulated. Alternatively or additionally, a bed, blanket or eiderdown filled with a gas/liquid, as well as a waterbed or air bed could be used of which the temperature can be controlled.

The computer arrangement 2 may, in an embodiment, also be programmed to generate its control signals for temperature manipulation units 242, 243 in dependence on output data from sleep monitoring device 4. Other temperature sensors 10(i) may be provided providing temperature data from other portions of the body, for instance, from a finger which has turned out to provide reliable temperature data indicating whether the subject is asleep or not. Data from other sleep monitoring devices may be used as well by computer arrangement 2 in this respect.

Suitable methods of sleep monitoring by periodically or continuously measuring skin temperature of at least one region of the subject's body have been described herein before with reference to FIGS. 1a, 1b, 2 and 3. It has been shown that temperature measurements at distal sites of the body provide reliable results. Therefore, in accordance with an aspect of the invention, it is preferred to acquire skin temperature data of more than one distal region in the subject's body. Typically, the skin temperature of one or more regions selected from any portion of the head, arms and legs, including hands and feet is measured. Preferably the distal region is selected from regions of an a lower arm, a wrist, a hand, a finger, an a lower leg, an ankle, a foot and toe of the subject, more preferably from regions of a lower arm, wrist, hand, finger, lower leg, ankle, foot and toe, most preferably from regions from the wrist and fingers.

In a suitable embodiment of the invention, the determination of the subject's state of sleep is made by computer arrangement 2 by using additional parameters that are known in the art to be indicative of sleep. One suitable parameter is the subject's motor activity which can be measured by motion detector 41. Methods of and arrangements for making sleep/wake classification based on a combination of motor activity data and skin temperature data has been described herein before. Other parameters that may be used by computer arrangement 2, are the ones as published by Raymann (2008), like. environmental characteristics including age, diagnosis, gender and environmental temperature.

### Manipulating Alertness.

The present inventors have also found that the determinations of a subject's level of alertness, as obtained in accordance with the above described method, can suitably be used in a method of improving or controlling alertness or alertness. Improving or controlling alertness may be desirable under circumstances wherein a subject is performing tasks which require a certain minimum level of alertness, especially if failure to perform such tasks involve the risk of said subject causing damage to itself and/or its surroundings, e.g. if the subject is operating a vehicle or heavy machinery, or performing monitoring tasks in critical environments.

The present inventors have found that it is possible to promote alertness by subtle manipulation of the trunks skin temperature. The mechanism underlying these findings is unknown. Without wishing to be bound by any theory, the present inventors hypothesize that certain brain areas involved in the regulation of alertness are sensitive to the trunk skin temperature and/or trunk skin temperature alterations.

Hence in a further aspect of the invention, a method is provided for controlling or improving a subject's level of alertness, comprising i) periodically or continuously measuring trunk skin temperature, and ii) manipulating the trunk skin temperature to remain within a predetermined temperature range such as to control alertness.

Suitable methods of monitoring a subject's trunk skin temperature and, in particular, methods of determining the subject's level of alertness on the basis of said trunk skin temperature, have been described in more detail herein before.

For a setup for an arrangement that can be used to monitor and control alertness, reference is made again to FIG. 6 which also shows a temperature control unit 59. The temperature control unit 59 is connected to and controlled by suitable control signals from computer arrangement 2. Temperature control unit 59 is designed to control the temperature of the trunk of the human body.

Several factors may determine the optimal site of the trunk for skin temperature manipulation, for example, body position (supine, sitting, standing), physical activity history, age, gender, body weight, sleep history, time awake, time of day, individual circadian phase, environmental temperature and humidity, environmental light and their interactions. Environmental temperature can be measured by temperature sensor 58(j). Other suitable sensors may be provided to measure the level of humidity, the level of environmental light, etc. If so, the computer arrangement 2 is programmed to receive output data of such sensors and to take these output data into account when assessing whether the person concerned is alert or not. Where appropriate, a temperature control unit arranged to control the environmental temperature at a predetermined level may be provided, as well as a humidity control unit to control environmental humidity level and a light control unit to control the level of ambient light. Such control unit may or may not be controlled by computer arrangement 2.

In an embodiment, the temperature sensors 10(i) are implemented as microsensors with integrated temperature recording and wireless signal transmission properties that can be attached to the skin of the user's trunk with adhesive tape or bands. The transmitted signals can be received with the computer arrangement 2.

In another embodiment, the temperature sensors 10(i) are implemented as a grid of miniature temperature sensors that can be integrated in the chair 22 beneath and/or at the back of the user, and/or the safety belt as present in a car.

During normal use of the chair 22, only a restricted selection of all temperature sensors 10(i) will be close enough to the skin of the user to provide a reliable estimate of skin temperature. Two methods may be applied alone or in parallel to aid automated selection of these temperature sensors 10(i). First, the temperature sensors 10(i) measuring the highest temperature are the temperature sensors 10(i) most likely to be in good contact with the skin, and their output data will be selected by computer arrangement 2 to establish whether the user is alert or not. Second, a grid of pressure sensitive sensors may be integrated with the grid of temperature sensors 10(i). At those sites where the pressure is highest, close contact with the skin is most likely. The output from temperature sensors 10(i) at those locations will be selected by computer arrangement 2 to establish whether the user is alert or not.

The computer arrangement 2 integrates information of the (selected) temperature sensors 10(i), for instance in a region-specific way (e.g. trunk, legs), compares this information with default and individualized optimal temperature settings as stored in memory 5, 7, 9, 11, and controls the temperature control unit 59.

The temperature control unit 59 may have a grid of temperature actuators integrated in chair 22 at the back of the person to change chair temperature. Actuators may be implemented either with thermo-electric devices for heating (e.g. heating elements) and cooling (e.g. peltier elements), or by control of valves of a tubing system through which temperature-conditioned gas or liquid can be circulated.

It is understood that the examples and embodiments described herein are for illustrative purposes only and that various modifications or changes in light thereof will be suggested to persons skilled in the art and are to be included within the spirit and purview of this application and scope of the appended claims. For instance, the sleep/alertness monitoring/manipulating arrangements can be combined in a single arrangement. All publications, patents, and patent applications cited herein are hereby incorporated by reference for all purposes.

1. A method of monitoring sleep in a subject comprising measuring within a prescribed interval skin temperature of a predetermined region of the subject's body and comparing said measured skin temperature of said predetermined region with a predetermined temperature threshold, measuring motor activity of said subject's body in said interval and classifying said subject as being asleep or being awake based on whether the skin temperature of said predetermined region is above or below said temperature threshold and on said motor activity.

2. The method according to claim 1, wherein the predetermined region is at least one distal region of the body and selected from a lower arm, a wrist, a hand, a finger, a lower leg, an ankle, a foot and a toe.

3. The method according to claim 2, wherein said temperature threshold is personalized and has a value within a range of 33.3° C. and 33.7° C.

4. The method according to claim 1, wherein motor activity of the subject is measured by sensing temporal motion of at least one part of the subject's body, comprising at least one of a part of a subject's arm, a part of a leg, and a wrist.

5. The method according to claim 4, wherein the skin temperature is measured with a temperature sensor at a wrist of said subject's human body and said motor activity is also measured at said wrist by means of a motion detector, said motion detector and temperature sensor being integrated in a single unit that can be worn by the subject on the subject's wrist.

6. The method according to claim 1, wherein said temperature threshold is established taking into account whether the subject is a person suffering from a sleeping disorder, an elderly person, or an adult having no sleeping disorder.

7. A sleep monitoring arrangement comprising at least one temperature sensor for measuring within a prescribed interval skin temperature of a predetermined region of a subject's body and to render measured skin temperature data, a motion detector for measuring motor activity of said subject's body in said interval and a processor arranged to receive said measured skin temperature data of said predetermined region as well as said motor activity, a memory storing a predetermined temperature threshold and a computer program with instructions and data allowing said processor to compare said measured skin temperature of said predetermined region with said predetermined temperature threshold and classify said subject as being asleep or being awake based on whether the skin temperature of said predetermined region is above or below said temperature threshold and on said motor activity.

8. The sleep monitoring arrangement according to claim 7, wherein said at least one temperature sensor is arranged to measure skin temperature in at least one of a lower arm, a wrist, a hand, a finger, a lower leg, an ankle, a foot and a toe.

9. The sleep monitoring arrangement according to claim 8, wherein said at least one temperature sensor and said motion detector are integrated in a single unit that can be attached to a wrist.

10. The sleep monitoring arrangement according to claim 9, wherein said single unit comprises at least one of a display, an input unit and a transceiver.

11. A method of promoting sleep in a subject, the method comprising controlling skin temperature of one or more selected portions of said body within a predetermined temperature range associated with a state of sleep of said subject.

12. The method according to claim 11, wherein the method comprises controlling skin temperature in the subject, such as to clamp the skin temperature 0.2-0.8° C. higher as compared to the skin temperature of the selected portion observed without or prior to manipulation under normal sleeping circumstances.

13. The method according to claim 11, wherein the method comprises proximal and distal skin warming.

14. (canceled)

15. The method according to claim 11, wherein said controlling said skin temperature uses at least one of the following relations:
distal skin warming enhances REM sleep and suppresses light sleep in young and older subjects without sleep complaints,
proximal warming results in deeper sleep and suppressed wakefulness,
in elderly insomniacs, proximal warming enhances slow wave and REM sleep whereas distal warming enhances slow wave sleep and suppresses REM sleep,
suitable mild skin temperature manipulations can significantly reduce early morning awakening, and
suitable skin temperature manipulations can be chosen to enhance deeper sleep stages.

16. The method according to claim 11, wherein controlling said skin temperature is performed while using at least one of the following parameters: body position, subject's age, subject's gender, subject's body weight, sleep history, time awake, time of day, subject's individual circadian phase, environmental temperature, environmental humidity, and environmental light.

17. A sleep manipulating arrangement comprising a computer arrangement and one or more temperature manipulation units, the computer arrangement being arranged to control said skin temperature of one or more selected portions of a subject's body within a predetermined temperature range associated with a state of sleep of said subject via said one or more temperature manipulation units.

18. The sleep manipulating arrangement according to claim 17, further comprising one or more temperature sensors arranged to measure skin temperature on the selected portion of said body and to send output signals to said computer arrangement.

19. (canceled)

20. The sleep manipulating arrangement according to claim 18, wherein the one or more temperature sensors are implemented as a grid of miniature temperature sensors that can be integrated in a bed.

21. The sleep manipulating arrangement according to claim 18, wherein the computer arrangement is arranged to use temperature data of only a selected portion of said one or more temperature sensors to establish said skin temperature of said one or more selected portions of said body by selecting temperature sensors measuring the highest temperature.

22. The sleep manipulating arrangement according to claim 18, wherein one or more pressure sensitive sensors are integrated with the one or more temperature sensors and the computer arrangement is arranged to use temperature data of only a selected portion of said one or more temperature sensors to establish said skin temperature of said one or more selected portions of said body by selecting temperature sensors at those sites where the pressure is highest.

23. The sleep manipulating arrangement according to claim 18, wherein the computer arrangement is arranged to integrate information of the one or more temperature sensors in a region-specific way, to compare this information with default and individualized optimal temperature settings, and to control the temperature manipulation units accordingly.

24. The sleep manipulating arrangement according to claim 17, wherein the temperature manipulation unit comprises at least one of:
actuators implemented either with thermoelectric devices for heating and cooling, and
a tubing system allowing temperature-conditioned gas or liquid to be circulated,
a bed, blanket or eiderdown filled with a gas/liquid,
a waterbed or air bed of which the temperature can be controlled.

25. A method of monitoring alertness in a subject comprising continuously or periodically measuring skin temperature; wherein an increase in said skin temperature during an interval is indicative of a reduction in alertness during said interval.

26. The method according to claim 25, wherein the temperature is measured on a subclavicular region or a region of a subject's chest.

27. An alertness monitoring arrangement comprising at least one temperature sensor for measuring within a prescribed time interval skin temperature and to render measured skin temperature data, a processor arranged to receive said measured skin temperature data, a memory storing a predetermined temperature threshold and a computer program with instructions and data allowing said processor to compare said measured skin temperature data with said predetermined temperature threshold, and to classify said subject as being alert if the measured skin temperature is below said temperature threshold and as being non-alert if the measured skin temperature is above said temperature threshold.

28. The alertness monitoring arrangement according to claim 27, wherein said at least one temperature sensor is arranged to measure said skin temperature on a trunk of the subject.

29. A method of manipulating a level of alertness of a subject, comprising i) continuously or periodically measuring skin temperature, and ii) manipulating a skin temperature such as to remain within a predetermined temperature range associated with a predetermined level of alertness.

30. (canceled)

31. An alertness manipulating arrangement arranged to perform a method according to claim 29, comprising an alertness monitoring arrangement to determine a level of alertness of a subject, and a temperature control unit to manipulate a skin temperature of a subject in order to control said subject to be alert.

32. The alertness manipulating arrangement according to claim 31, comprising a steering wheel for a car, the steering wheel having integrated temperature sensors to measure said skin temperature.

33. An arrangement comprising a combination of either:
a sleep monitoring arrangement according to claim 7 and an alertness monitoring arrangement according to claim 27, or
a sleep manipulating arrangement according to claim 17 and an alertness manipulating arrangement according to claim 31.

34. The method according to claim 12, wherein the method comprises proximal and distal skin warming.

35. The method according to claim 12, wherein controlling said skin temperature is performed while using at least one of the following parameters: body position, subject's age, subject's gender, subject's body weight sleep history, time awake, time of day, subject's individual circadian phase, environmental temperature, environmental humidity, and environmental light.
